(19) United States
(12) Patent Application Publication
(10) Pub. No.: US 2021/0283049 A1
(43) Pub. Date: Sep. 16, 2021

(54) INTRAVENOUS SOTALOL HYDROCHLORIDE LOADING AND MAINTENANCE FOR CARDIAC SURGERY PATIENTS

(71) Applicant: AltaThera Pharmaceuticals LLC, Chicago, IL (US)

(72) Inventor: John Somberg, Chicago, IL (US)

(21) Appl. No.: 17/306,490

(22) Filed: May 3, 2021

Related U.S. Application Data
(63) Continuation of application No. 16/849,099, filed on Apr. 15, 2020.
Application No. 16/849,099 is a continuation-in-part of application No. 16/693,312, filed on Nov. 24, 2019.
Application No. 16/693,312 is a continuation of application No. 16/103,815, filed on Aug. 14, 2018, now Pat. No. 10,512,620.
Application No. 16/103,815 is a continuation-in-part of application No. 16/693,310, filed on Nov. 24, 2019.
Application No. 16/693,310 is a continuation-in-part of application No. 16/103,815, filed on Aug. 14, 2018, now Pat. No. 10,512,620.
(60) Provisional application No. 62/987,832, filed on Mar. 10, 2020.

(51) Int. Cl. A61K 9/00; A61K 31/145

(52) U.S. Cl. CPC A61K 9/0019; A61K 31/145

(57) ABSTRACT

The present invention provides a novel intravenous, prophylactic, antiarrhythmic method of sotalol loading and maintenance for cardiothoracic surgery patients.

### CROSS-REFERENCE TO RELATED APPLICATIONS

This application is a Continuation application of U.S. application Ser. No. 16/849,099, filed Apr. 15, 2020, which relies on the disclosure of and claims priority to and the benefit of the filing date of U.S. Provisional Application No. 62/987,832, filed Mar. 10, 2020. This application is a Continuation-in-Part (CIP) application of U.S. application Ser. Nos. 16/693,310 and 16/693,312, filed Nov. 24, 2019. The '310 application is a CIP of and the '312 application is a Continuation of U.S. application Ser. No. 16/103,815, filed Aug. 14, 2018, which issued as U.S. Pat. No. 10,512,620 on Dec. 24, 2019. The disclosure of each of these applications is hereby incorporated by reference herein in their entireties.

### FIELD OF THE INVENTION

The present invention provides a novel method of intravenously loading and maintaining sotalol hydrochloride in cardiothoracic surgery patients.

### BACKGROUND OF THE INVENTION

Patients who have a history of atrial fibrillation (AF) and/or atrial flutter (AFL) (AF/AFL) and who are not currently on antiarrhythmic therapy, but who are scheduled for cardiothoracic surgery (e.g., open heart surgery or coronary angioplasty), are often at risk for AF/AFL developing during or just after the cardiothoracic surgery. If patients develop AF/AFL during surgery or post-surgery, then a prolongation of their hospital stays (compared to similar patients not developing AF/AFL) will occur. In addition, clinical trials on post-surgery AF increase overall mortality versus patients who do not develop AF following surgery. It would be beneficial for both the hospital (#of beds available is limited) and patient (cost and inconvenience of a long stay as well as the life-threatening risks of AF/AFL) if AF/AFL could be prevented or at least the risk of it reduced during cardiothoracic surgery.

Gomes et al. (JACC 1999, 34(2), 334-339) (Gomes) described how oral sotalol (PO sotalol) reduces the incidence of AF post coronary artery bypass surgery (CABG) (67% decrease in postoperative AF for patients receiving PO sotalol). The patients in this study were not the highest risk patients as they did not have a history of AF.

Because sotalol increases the QT interval and this increase can cause or worsen arrhythmias, patients must be observed in hospital while sotalol is loaded. When this is done orally, 3 days are required for hospital observation (see oral sotalol label). Thus, PO sotalol, while useful for reducing postoperative AF for CABG patients (without a history of AF), comes with the additional expense and inconvenience of a 3-day preoperative hospitalization.

It would be beneficial to discover a method of administering antiarrhythmic therapy that reduces the peri- and postoperative risk of AF and/or AFL without a concomitant increase of preoperative hospitalization.

### SUMMARY OF THE INVENTION

Accordingly, in an aspect, the present invention provides a novel intravenous, prophylactic, antiarrhythmic method, comprising: intravenously loading and maintaining a therapeutically effective amount of sotalol hydrochloride in a cardiothoracic surgery patient.

In another aspect, the present invention provides a novel intravenous, prophylactic, antiarrhythmic method, comprising: intravenously loading and maintaining a therapeutically effective amount of sotalol hydrochloride in a cardiothoracic surgery patient who has a history of AF and/or AFL and who is not currently on antiarrhythmic therapy.

In another aspect, the present invention provides a novel intravenous, prophylactic, antiarrhythmic method that reduces the risk of AF and/or AFL in cardiothoracic surgery patients, comprising: intravenously loading and maintaining a therapeutically effective amount of sotalol hydrochloride in the patient.

In another aspect, the present invention provides a novel intravenous, prophylactic, antiarrhythmic method that shortens the length of a cardiothoracic surgery patient's hospital stay, comprising: intravenously loading and maintaining a therapeutically effective amount of sotalol hydrochloride in the patient.

These and other objects, which will become apparent during the following detailed description, have been achieved by the inventors' discovery that sotalol can be intravenously loaded and maintained in a cardiothoracic surgery patient.

### DETAILED DESCRIPTION OF THE INVENTION

All references cited herein are hereby incorporated in their entirety herein by reference.

AF is atrial fibrillation.

AFL is atrial flutter.

AF/AFL=atrial fibrillation and/or atrial flutter.

IV is intravenous.

PO means “per os” and refers to an oral dosing regimen.

BID means “bis in die” and means twice a day.

Patient (or subject) refers to a human patient.

BP is blood pressure.

HR is heart rate.

QT is the interval measured from the start of the Q wave or the QRS complex, to the end of the T wave, where the Q wave corresponds to the beginning of ventricular depolarization and the T wave end corresponds to the end of ventricular repolarization.

QTc is the calculated interval that represents the QT interval corrected for heart rate and can be derived by simple mathematical correlation of the QT interval and the heart rate.

ΔQTc is the difference between a QTc measurement taken prior to the start of IV sotalol and a QTc measured after the start of IV sotalol (e.g., during loading or maintenance).

Sotalol and sotalol hydrochloride (used interchangeably herein) refer to d,l-sotalol hydrochloride which has been approved by the FDA for intravenous administration over 5 hours or oral administration (e.g., 80 mg, 120 mg, and 160 mg tablets).

Cardiothoracic surgery patient is a patient who has been diagnosed with a condition that requires cardiothoracic surgery and for whom surgery has been scheduled with a hospital. Cardiothoracic surgeries include both open and minimally invasive cardiothoracic surgeries that pose risk for AF/AFL. Examples of open cardiothoracic surgeries include coronary arterial bypass graft surgery (CABG), bypass graft surgery, valve replacement surgery, combined CABG and valve surgeries, thoracic aneurysm surgery, heart transplant, lung transplant, thoracotomy surgery (lobectomy, wedge resections, pneumonectomy, etc.), and esophageal surgery. Examples of minimally invasive cardiac procedures include coronary angioplasty, transcatheter aortic valve replacement (TAVR), cardiac stenting, mitral valve repair, complex aortic procedures, aortic valve repair and replacement, mitral valve replacement, robotic mitral valve surgery, mini-maze surgery for atrial fibrillation, endovascular procedures, video-assisted thoracic surgery (VATs), and robotic thoracic surgery.

Preoperative refers to a cardiothoracic surgery patient who has been admitted to a hospital for cardiothoracic surgery, but the surgery has not yet begun.

Perioperative refers to a cardiothoracic surgery patient who is hospitalized and undergoing cardiothoracic surgery.

Postoperative refers to a cardiothoracic surgery patient who is hospitalized and has undergone cardiothoracic surgery.

Hospital refers to a medical facility staffed and equipped for cardiac surgeries and can provide continuous ECG monitoring and cardiac resuscitation to patients, if needed.

Shortening the length of a hospital stay refers to the length of time a patient is admitted for preoperative processing until the time the patient is postoperatively discharged. The shortening is as compared with the same cardiothoracic surgery without the present prophylactic, antiarrhythmic method.

Reducing the risk of AF and/or AFL in cardiothoracic surgery patients refers to the risk of patients receiving the prophylactic, antiarrhythmic method of the present invention compared with the risk of those not treated with the prophylactic, antiarrhythmic method of the present invention.

Therapeutically effective amount refers to an amount of sotalol that will reduce the perioperative and/or postoperative risk of AF and/or AFL of a cardiothoracic surgery patient.

As noted above, the study reported by Gomes used PO sotalol demonstrates that the risk of AF in patients without a history of AF can be reduced for coronary artery bypass surgery. With the current standard of a 3-day hospitalization required for sotalol initiation, the procedure of Gomes, while reducing risk for a certain class of patients, does not reduce hospitalization time (at least not significantly). For the class of cardiothoracic surgery patients who have a history of AF and/or AFL and who are not currently on antiarrhythmic therapy, there remains a need for a prophylactic, antiarrhythmic method to substantially reduce the risk of (or prevent) AF/AFL from developing during surgery. These patients would also benefit from a method that does not increase the number of hospitalization days, which is required for oral sotalol loading.

Thus, in an aspect, the present invention provides a novel intravenous, prophylactic, antiarrhythmic method for reducing the risk of a atrial fibrillation (AF), atrial flutter (AFL) or both in a cardiothoracic surgery patient, comprising: intravenously loading and maintaining a therapeutically effective amount of sotalol hydrochloride in the cardiothoracic surgery patient in need thereof.

In the present invention, the physician of a patient who is scheduled to have cardiothoracic surgery selects a dose of oral sotalol that would be targeted (e.g., 80 mg, 120 mg, or 160 mg) and then the IV load would be determined based on the targeted oral dosage. As shown in Table 1, the IV Load and Maintenance are based on the targeted oral sotalol dosage of 80, 120, or 160 mg (the current FDA approved oral dosages).

TABLE 1
IV Sotalol Load and Maintenance Protocol

| | | | |
|---|---|---|---|
| Target dose* (mg) | 80 | 120 | 160 |
| 1V Load (mg over 1 h) | 65 | 95 | 125 |
| Time to first dose (h) | 1 | 1 | 1 |
| IV Maintenance (mg over 5 h)** | 75 | 112.5 | 150 |

*Selected by patient's physician/surgeon.
**Every 12 h thereafter until discontinued (stopped or switched to oral BID)

The cardiothoracic surgery patient receives the selected IV load (e.g., 65 mg, 95 mg, or 125 mg) from up to 5 hours before the operation (preoperatively) to up to 5 hours after the operation (postoperatively). Examples of preoperative IV load include initiating the IV Load 5, 4, 3, 2, to 1 hour before surgery. In another example, the IV load is initiated perioperatively. Examples of the postoperative IV load include initiating the IV load 1, 2, 3, 4 to 5 hours after surgery.

The IV load, as shown in Table 1, is typically administered over 1 hour. Additional examples of the time over which the IV load is administered, include 50-70 minutes. Further examples include 50, 51, 52, 53, 54, 55, 56, 57, 58, 59, 60, 61, 62, 63, 64, 65, 66, 67, 68, 69, and 70 minutes.

Additional examples of the IV load for the target dose of 80 mg include 55-85 mg. Further examples include 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, and 85 mg.

Additional examples of the IV load for the target dose of 120 mg include 75-115 mg. Further examples include 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, and 115 mg.

Additional examples of the IV load for the target dose of 160 mg include 100-150 mg. Further examples include 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 mg.

One hour after completion of the IV Load (or two hours after initiation) the corresponding first IV Maintenance dose (75 mg, 112.5 mg, or 150 mg) is started. The IV Maintenance dose is delivered over 5 h (the current standard of therapy for IV sotalol).

Additional examples of the time over which the IV maintenance dose (or dosages) is administered include 3-6 hours. Further examples include 180, 190, 200, 210, 220, 230, 240, 250, 260, 270, 280, 290, 300, 310, 320, 330, 340, 350, and 360 minutes.

Additional examples of the IV maintenance for the target dose of 80 mg include 60-90 mg. Further examples include 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and 90 mg.

Additional examples of the IV maintenance for the target dose of 120 mg include 100-120 mg. Further examples include 100, 101, 102, 103, 104, 105, 106, 107, 108, 109, 110, 111, 112, 113, 114, 115, 116, 117, 118, 119, and 120 mg.

Additional examples of the IV maintenance for the target dose of 160 mg include 135-165 mg. Further examples include 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, and 165 mg.

In another aspect, the IV maintenance dose is initiated 0.5-5 hours after completion of the IV load. Further examples include from 30, 45, 60, 75, 90, 105, 120, 135, 150, 165, 180, 195, 210, 225, 240, 255, 270, 285, to 300 minutes after completion of the IV load.

In another aspect, the IV maintenance is administered once every 12 hours.

In another aspect, after surgery, the IV maintenance dosing is continued, stopped, or switched to the corresponding oral sotalol dose BID (every 12 hours) depending on the patient's condition.

In another aspect, the IV maintenance doses are continued for 1, 1.5, 2, 2.5, to 3 days postoperatively (once every 12 h or 2 IV doses per 24 h).

In another aspect, the patient is switched to oral sotalol BID once the patient can take oral medications. The oral therapy is maintained at the patient's physician's discretion. For example, the oral therapy can be continued 1, 2, 3, 4, 5, 6, 7, 8, 9, 10, 11, 12, 13, 14, or more days postoperatively. Oral therapy can be continued after the patient has been discharged from the hospital.

In another aspect, the present invention provides a novel intravenous, prophylactic, antiarrhythmic method that reduces the risk of AF and/or AFL in cardiothoracic surgery patients, comprising: intravenously loading and maintaining a therapeutically effective amount of sotalol hydrochloride in a cardiothoracic surgery patient.

In another aspect, the present invention provides a novel intravenous, prophylactic, antiarrhythmic method that reduces the risk of AF and/or AFL in cardiothoracic surgery patients and that shortens the length of a cardiac patient's hospital stay, comprising: intravenously loading and maintaining a therapeutically effective amount of sotalol hydrochloride in a cardiothoracic surgery patient.

In another aspect, the present invention provides a novel intravenous, prophylactic, antiarrhythmic method that shortens the length of a cardiothoracic surgery patient's hospital stay, comprising: intravenously loading and maintaining a therapeutically effective amount of sotalol hydrochloride in a cardiothoracic surgery patient.

In another aspect, the method reduces the perioperative and postoperative risk of AF and/or AFL of the cardiothoracic surgery patient.

In another aspect, the method reduces the perioperative and postoperative risk of AF and/or AFL of the cardiothoracic surgery patient without an increase of postoperative hospitalization.

In another aspect, the method reduces the postoperative hospitalization of the patient.

In another aspect, the method reduces the overall time of hospitalization of the patient.

In another aspect, the cardiothoracic surgery patient has a history of AF and/or AFL and is not currently on antiarrhythmic therapy.

In another aspect the IV loading dose is 65 mg and the IV maintenance dose is 75 mg.

In another aspect the IV loading dose is 95 mg and the IV maintenance dose is 112.5 mg.

In another aspect the IV loading dose is 125 mg and the IV maintenance dose is 150 mg.

In another aspect, the IV load is initiated preoperatively.

In another aspect, the IV load is initiated perioperatively.

In another aspect, the IV load is initiated postoperatively.

In another aspect, the IV maintenance occurs preoperatively.

In another aspect, the IV maintenance occurs perioperatively.

In another aspect, the IV maintenance occurs preoperatively and perioperatively.

In another aspect, the IV maintenance continues postoperatively.

In another aspect, the IV maintenance occurs perioperatively and postoperatively.

In another aspect, the IV maintenance occurs preoperatively and perioperatively and continues postoperatively.

In another aspect, the IV load is initiated preoperatively and the IV maintenance occurs preoperatively and perioperatively.

In another aspect, the IV load is initiated preoperatively and the IV maintenance occurs preoperatively, perioperatively, and postoperatively.

In another aspect, the IV load is initiated preoperatively and the IV maintenance occurs perioperatively.

In another aspect, the IV load is initiated preoperatively and the IV maintenance occurs perioperatively and post operatively.

In another aspect, the IV load is initiated perioperatively and the IV maintenance occurs postoperatively.

In another aspect, the IV load is initiated perioperatively and the IV maintenance occurs perioperatively and postoperatively.

In another aspect, the IV load is initiated postoperatively and the IV maintenance occurs postoperatively.

In another aspect, the patient's QTc is monitored via electrocardiography.

In another aspect, the patient's QTc is measured at baseline (prior to sotalol administration) and then measured periodically thereafter (e.g., every 15 or 30 minutes during loading). The QTc can be measured at other intervals if more (shorter time period) or less data (longer time periods) data is desired. The QTc is also typically monitored for 15-30 minutes after completion of the IV administration.

In another aspect, the patient's QTc interval is monitored every 15 (or 30) minutes after intravenous sotalol hydrochloride initiation (and throughout the time the patient remains on IV sotalol). If the patient's QTc is observed to be greater than 500 msec or if the ΔQTc is greater than or equal to 20% of the subject's QTc prior to sotalol administration, the IV sotalol is discontinued.

In another aspect, the HR and BP the patient is monitored every 15 minutes (or 30 minutes) during IV administration. If a BP below 90 mmHg and HR <50 bpm are observed, then the IV is discontinued. The HR and BP are also typically monitored for 15-30 minutes after completion of the IV administration.

The present invention may be embodied in other specific forms without departing from the spirit or essential attributes thereof. This invention encompasses all combinations of aspects of the invention noted herein. It is understood that any and all embodiments of the present invention may be taken in conjunction with any other embodiment or embodiments to describe additional embodiments. It is also to be understood that each individual element of the embodiments is intended to be taken individually as its own independent embodiment. Furthermore, any element of an embodiment is meant to be combined with any and all other elements from any embodiment to describe an additional embodiment.

Other features of the invention will become apparent in the course of the following descriptions of exemplary embodiments that are given for illustration of the invention and are not intended to be limiting thereof.

### EXAMPLES

### Example 1

A male patient, age 60, diagnosed with 3-vessel coronary artery disease—is scheduled for open heart surgery. Twelve hours before surgery the patient is admitted to the hospital. 1 hour prior to the start of surgery the patient is connected to an electrocardiograph and then the patient is loaded with 65 mg of sotalol via a 1-hour IV infusion. The patient's QT is measured prior to the IV start and monitored every 15 minutes during the IV load along with HR and BP. The patient's QTc is calculated from the QT measurement. If the patient's QTc is observed to be greater than 500 msec or if the ΔQTc is greater than or equal to 20% of the subject's QTc prior to sotalol administration, the IV sotalol is discontinued.

One hour after completion of the IV load, the patient receives 75 mg of IV sotalol via a 5-hour infusion (IV maintenance). The patient's QT is monitored at baseline and every hour thereafter as with the IV load. The maintenance dose is discontinued if the patient's QTc is observed to be greater than 500 msec or if the ΔQTc that is greater than or equal to 20% of the subject's QTc prior to sotalol administration.

The infusion is continued through surgery and repeated every 12 hours until 2 days post operation.

Numerous modifications and variations of the present invention are possible considering the above teachings. It is therefore to be understood that within the scope of the appended claims, the invention may be practiced otherwise than as specifically described herein.

What is claimed is:

1. An intravenous, prophylactic, antiarrhythmic method for reducing the risk of an atrial fibrillation (AF), atrial flutter (AFL) or both in a cardiothoracic surgery patient, comprising:
intravenously administering sotalol hydrochloride in a cardiothoracic surgery patient for maintenance of normal sinus rhythm;
wherein one of three dosing regimens (a)-(c) is followed for the patient:
a. an IV loading dose based on an oral target of 80 mg and selected from 55-85 mg and one or more subsequent IV doses selected from 60-90 mg;
b. an IV loading dose based on an oral target of 120 mg and selected from 75-115 mg and one or more subsequent IV doses selected from 100-120 mg; or
c. an IV loading dose based on an oral target of 160 mg and selected from 100-150 mg and one or more subsequent IV doses selected from 135-165 mg;
wherein the IV loading dose is a 1 hour infusion;
wherein one or more of the subsequent IV doses is administered by way of an infusion of up to 5 hours; and
wherein an interval between two of the one or more subsequent IV doses is an interval of 12-48 hours.

2. The method of claim 1, wherein one of three dosing regimens (a)-(c) is followed for the patient:
a. the IV loading dose is based on an oral target of 80 mg and is 65 mg and one or more of the subsequent IV doses is selected from 60-90 mg;
b. the IV loading dose is based on an oral target of 120 mg and is 95 mg and one or more of the subsequent IV doses is selected from 100-120 mg; or
c. the IV loading dose is based on an oral target of 160 mg and is 125 mg and one or more of the subsequent IV doses is selected from 135-165 mg.

3. The method of claim 1, wherein one of three dosing regimens (a)-(c) is followed for the patient:
a. the IV loading dose is based on an oral target of 80 mg and is 65 mg and one or more of the subsequent IV doses is 75 mg;
b. the IV loading dose is based on an oral target of 120 mg and is 95 mg and one or more of the subsequent IV doses is 112.5 mg; or
c. the IV loading dose is based on an oral target of 160 mg and is 125 mg and one or more of the subsequent IV doses is selected from 150 mg.

4. The method of claim 1, wherein one of three dosing regimens (a)-(c) is followed for the patient:
a. the IV loading dose is based on an oral target of 80 mg and is selected from 55-85 mg and one or more of the subsequent IV doses is 75 mg;
b. the IV loading dose is based on an oral target of 120 mg and is selected from 75-115 mg and one or more of the subsequent IV doses is 112.5 mg; or
c. the IV loading dose is based on an oral target of 160 mg and is selected from 100-150 mg and one or more of the subsequent IV doses is 150 mg.

5. The method of claim 1, wherein dosing regimen (a) is followed.

6. The method of claim 2, wherein dosing regimen (a) is followed.

7. The method of claim 3, wherein dosing regimen (a) is followed.

8. The method of claim 4, wherein dosing regimen (a) is followed.

9. The method of claim 1, wherein dosing regimen (b) is followed.

10. The method of claim 2, wherein dosing regimen (b) is followed.

11. The method of claim 3, wherein dosing regimen (b) is followed.

12. The method of claim 4, wherein dosing regimen (b) is followed.

13. The method of claim 1, wherein dosing regimen (c) is followed.

14. The method of claim 2, wherein dosing regimen (c) is followed.

15. The method of claim 3, wherein dosing regimen (c) is followed.

16. The method of claim 4, wherein dosing regimen (c) is followed.

17. The method of claim 1, wherein a minimum delay to a first of the one or more subsequent IV doses is 0.5-5 hours.

18. The method of claim 1, wherein:
a. the interval is 12-24 hours; and
b. a minimum delay to a first of the one or more subsequent IV doses is 1-6 hours.

19. The method of claim 1, wherein:
a. the interval is 12 hours; and
b. a minimum delay to a first of the subsequent IV doses is 1-4 hours.

20. The method of claim 1, wherein:
a. the interval is 24-48 hours; and
b. a minimum delay to a first of the subsequent IV doses is 6-12 hours.
